(19) United States
(12) Patent Application Publication
(10) Pub. No.: US 2022/0401087 A1
(43) Pub. Date: Dec. 22, 2022

(54) SYSTEMS AND METHODS FOR TREATMENTING THE PATENT FOREMAN OVALE AND ATRIAL SEPTAL DEFECT

(71) Applicant: Ventrimend, Inc, Lake Forest, CA (US)

(72) Inventors: David Zarbatany, Lake Forest, CA (US); Ricardo Roman, Lake Forest, CA (US)

(73) Assignee: Ventrimend, Inc, Lake Forest, CA (US)

(21) Appl. No.: 17/846,405

(22) Filed: Jun. 22, 2022

Related U.S. Application Data
(60) Provisional application No. 63/213,624, filed on Jun. 22, 2021.

(51) Int. Cl. A61B 17/00

(52) U.S. Cl. CPC A61B 17/0057; A61B 2017/00592; A61B 2017/00619; A61B 2017/00867; A61B 2017/00623

(57) ABSTRACT

The present invention describes systems and methods for treating a hole in the septum, such as a patent foramen ovale or an atrial septal defect, with a closure implant designed to fluidly seal the hole to prevent blood from flowing between the right atrium to the left atrium. The closure implant includes two structures positioned on opposite sides of the hole in septum configured to close and seal the hole. The first structure is an expandable petal anchor having multiple petal groups with multiple petals of different sizes and/or shapes made of shape memory wire configured to be positioned on a distal side of the hole in the septum wall, and the second structure is a braided disk made of shape memory mesh positioned the proximal side of the hole in the septum wall, opposite the petal anchor. The braided disk is sized to cover the hole once it is expanded. The petal anchor is designed to pull and compress the braided disk against the septum wall to cover and fluidly seal the hole. The closure implant is designed to be delivered to the heart using a catheter-based delivery system.

### CROSS-REFERENCE TO RELATED APPLICATIONS

This application claims priority to U.S. Provisional Patent Application No. 63/213,624, filed Jun. 22, 2021, the entire disclosure of which is incorporated by reference herein.

### FIELD

The present invention relates generally to the field of surgery, and more specifically, to treatment of a patent foramen ovale and atrial septal defect.

### BACKGROUND

There are two kinds of holes in the septum of the heart, a patent foramen ovale and an atrial septal defect. Both are holes in septum between the left and right atrium. The patent foramen ovale is a hole that is left when the foramen ovale does not close after birth, and the atrial septal defect is a hole in the septum wall that is present at birth.

The foramen ovale is a small hole with a flap located in the septum wall between the right and left atriums of the heart. During fetal development, foramen ovale flap opens and allows blood from the mother to go from the veins to the right atrium of the heart, and through the foramen ovale directly to the left atrium of the heart. After birth, the blood flow through the heart is reversed and the flap on the foramen ovale normally closes as blood pressure rises in the left atrium of the heart. Once it is closed, the blood flows to the lungs to get oxygen before it enters the left atrium of the heart and gets pumped to the rest of the body.

In some people, the foramen ovale does not close properly, so there is still an opening in the septum. This condition is called a patent foramen ovale. Normally, the patent foramen ovale flap stays closed, but sometimes the flap may open when there is higher pressure than normal in the right atrium of the heart. Situations that can cause greater pressure include straining during bowel movements, coughing and sneezing. When the pressure gets high enough, blood may move from the right atrium to the left atrium.

An atrial septal defect is a hole in the septum wall between the right and left atriums of the heart. The condition is present at birth and small atrial septal defects may close during infancy or early childhood and never cause a problem. If the hole stays open, it may increase the amount of blood that flows through the lungs and cause damage the heart and lungs.

Both the patent foramen ovale and atrial septal defect increase the risk of transient ischemic attack, stroke and heart attack. This is because it is possible for a blood clot or solid particles in the blood to move from the right side of the heart to the left through the opening in the septum and travel to the brain or a coronary artery.

Accordingly, there remains a need for systems and methods that provide solutions to the patent foramen ovale and atrial septal defect problem. The present invention is directed toward systems and methods for treating these problems.

### SUMMARY

The present invention describes systems and methods for treating a hole in the septum, such as a patent foramen ovale or an atrial septal defect, with a closure implant designed to fluidly seal the hole to prevent blood from flowing between the right atrium to the left atrium. The closure implant includes two structures positioned on opposite sides of the hole in septum configured to close and seal the hole. The first structure is an expandable braided disk with fabric seal that is positioned on one side of the hole in the septum wall. The braided disk is sized to cover the hole once it is expanded. The second structure is an expandable petal anchor having asymmetrical petals (larger and smaller) positioned the other side of the hole in the septum wall, opposite the braided disk. The petal anchors are designed to pull and compress the braided disk against the septum wall to cover and fluidly seal the hole. The closure implant is designed to be delivered to the heart using a catheter-based delivery system.

### BRIEF DESCRIPTION OF THE DRAWINGS

FIG. 1 is a sectional view of the heart anatomy.

FIGS. 2A-2C are enlarged views showing different conditions of the septum wall.

FIG. 3 shows one embodiment of a closure implant designed to close holes or defects in the septum.

FIGS. 4A and 4B show the closure implant coupled to the distal end of a delivery system.

FIG. 5 shows the closure implant coupled to a delivery system.

FIG. 6A-6C shows various embodiments of a pedal anchor 110 having petal groups with asymmetrical petals.

FIGS. 7A-7H shows one embodiment of a closure implant that is delivered to an atrial septal defect through a delivery system in an interventional procedure.

FIG. 8 is a perspective showing the components of the connect/disconnect feature.

FIG. 9 is perspective view showing the closure implant coupled to the delivery system.

FIG. 10 is perspective view of the connect/disconnect feature of the invention showing the delivery system coupled to the closure implant.

FIG. 11 is a sectional view showing the engagement of the distal end of the shaft and engagement arms coupled to the coupler and slots.

FIG. 12 is a sectional view showing the delivery system disengaged from the closure implant.

### DETAILED DESCRIPTION

The present invention describes systems and methods for treating a patent foramen ovale or an atrial septal defect. The invention includes a closure implant designed to close a hole or defect in the septum to prevent blood from flowing between the right atrium to the left atrium. The closure implant includes two structures positioned on opposite sides of the hole in septum configured to close and seal the hole. The closure implant includes two structures positioned on opposite sides of the hole in septum configured to close and seal the hole. The first structure is an expandable braided disk with fabric seal that is positioned on one side of the hole in the septum wall. The braided disk is sized to cover the hole once it is expanded. The second structure is an expandable petal anchor having asymmetrical petals (larger and smaller) positioned the other side of the hole in the septum wall, opposite the braided disk. The petal anchors are designed to pull and compress the braided disk against the septum wall to cover and fluidly seal the hole. The closure implant is designed to be delivered to the heart using a catheter-based delivery system that has the ability to torque the asymmetrical petals for optimum seal and optimum durability. The delivery system also includes a connect/disconnect structure that is designed to allows torquing the implant into the desired position and zero release force when disconnected from the closure implant.

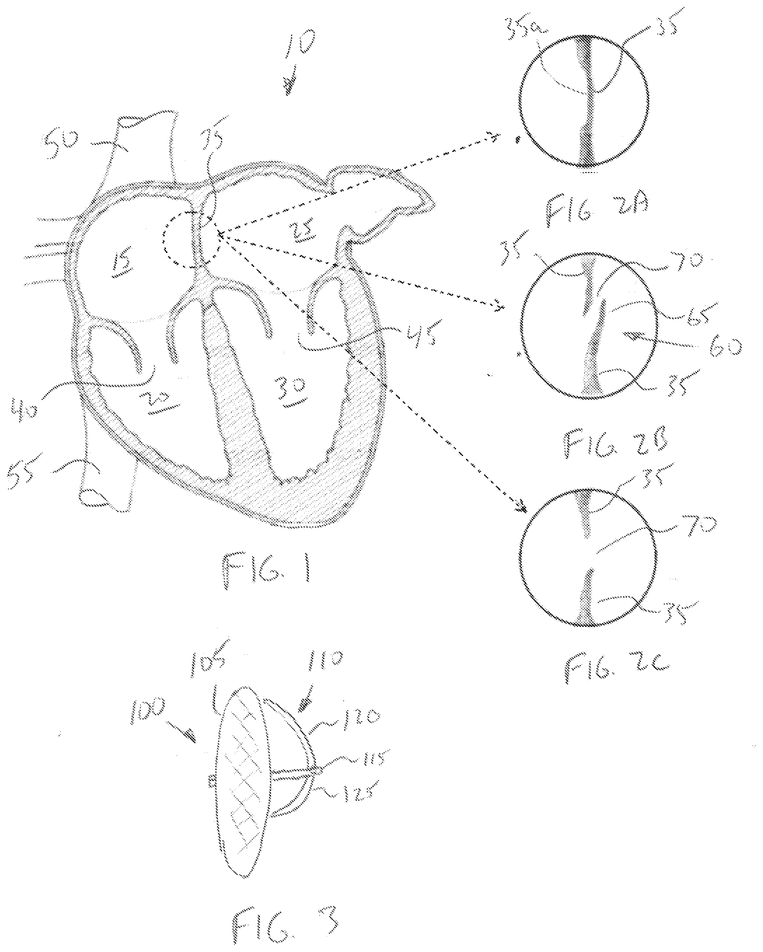

FIG. 1 is a sectional view showing the anatomy of a normal heart 10. The heart 10 includes four chambers, including a right atrium 15, a right ventricle 20, a left atrium 25 and a left ventricle 30. The right atrium 15 and left atrium 25 are separated by the atrial septum 35. A tricuspid valve 40 allows blood to flow from the right atrium 15 into the right ventricle 20. A mitral valve 45 allows blood to flow from the left atrium 25 into the left vertical 30.

Blood enters the right atrium 15 from the superior vena cava 50 and the inferior vena cava 55 blood vessels. The blood flows into the right atrium 15, through the tricuspid valve 40 into the right ventricle 20. Blood then flows from the right ventricle 20 into the pulmonary aorta to the lungs. Once through the lungs, the blood flows back to the heart and into the left atrium 25. The blood from the left atrium 25 flows through the mitral valve 45 into the left ventricle 30 and out of the heart to the ascending aorta.

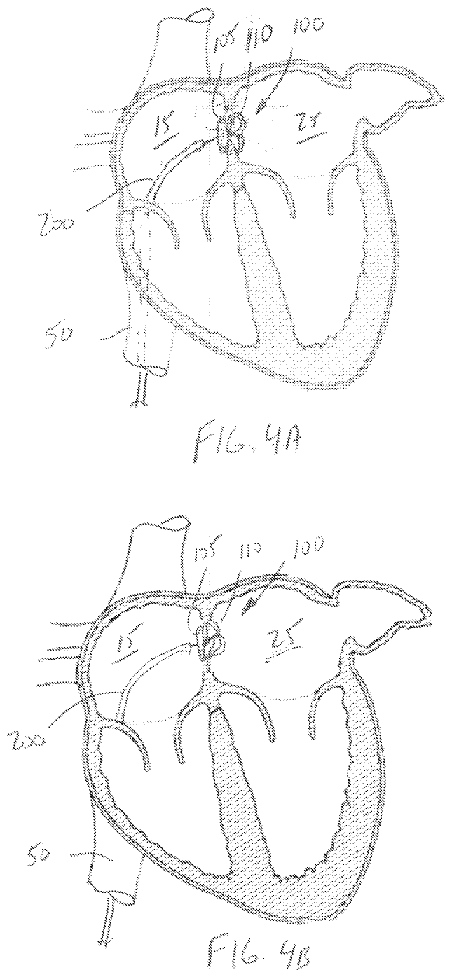

FIGS. 2A-2C are enlarged views showing different conditions that the septum wall might have.

FIG. 2A is an enlarged view of the septum 35 showing a fossa ovalis 35a. The fossa ovalis 35a is the remnant of a foramen ovale that was open at birth but then closed after birth. The fossa ovalis 35a appears as a depression in the septum 35.

FIB. 2B is an enlarged view of the septum 35 showing a foramen ovale that was open at birth but did not close, forming a patent foramen ovale 60 positioned between the right atrium 15 and left atrium 25. The patent foramen ovale 60 also includes a hole 70 and a flap 65 that is normally closed due to a higher pressure within the left atrium 25 than the pressure in the right atrium 15. In some cases, different events may increase the pressure in the right atrium 15 above the left atrium 25, allowing the flap 65 to open and blood to flow in reverse through the hole 70. This may lead to heart attacks, strokes, or other organ ischemia.

FIB. 2C is an enlarged view of the septum 35 showing an atrial septal defect 70 in the septum 35 between the right atrium 15 and the left atrium 25. The Atrial Septal Defect 70 allows oxygen-rich blood to leak or flow from the right atrium 15 and left atrium 25. The extra blood being pumped into the lung arteries makes the heart and lungs work harder and the lung arteries can become gradually damage.

FIG. 3 shows one embodiment of a closure implant 100 having a braided disk 105 and an asymmetrical petal anchor 110 positioned on an implant shaft 115. The braided disk 105 is designed to fluidly seal a hole in the septum between the right atrium 15 and the left atrium 25. The closure implant 100 can be implanted in a surgical procedure or via one or more catheter-based delivery systems in an interventional procedure.

The braided disk 105 includes an expandable mesh structure that is covered with a covering material to form a fabric seal. The braided disk 105 is self-expanding and is made from a shaped memory mesh, such as nitinol, that may be pre-shaped in a desired disk shape and configured to be collapsed or compressed shape within a delivery system. The braided disk 105 then self-expand after being delivered by the delivery system. The covering material may be a biocompatible material. In some embodiments, the covering material is selected from the group consisting of: a woven material; a fabric; a wire mesh; polyethylene terephthalate; a sponge; cellulose; synthetic fiber; cotton; rayon; hydrogel; a coagulant; a biodegradable material; a non-biodegradable material and combinations of one, two, or more of these.

In the embodiment shown, the asymmetrical petal anchor 115 includes asymmetrical petal groups 120, 125 arranged like the petals of a flower configured to engage the wall proximate the septum to anchor the closure implant 100. The petals are made of a shape-memory wire, such as a nitinol wire, that may be pre-shaped in a petal shape to allow the petals to be delivered in a collapsed or compressed shape within a delivery system. Then once delivered, shape-memory wire self-expands the petal back into the pre-shaped petal when released from the delivery system.

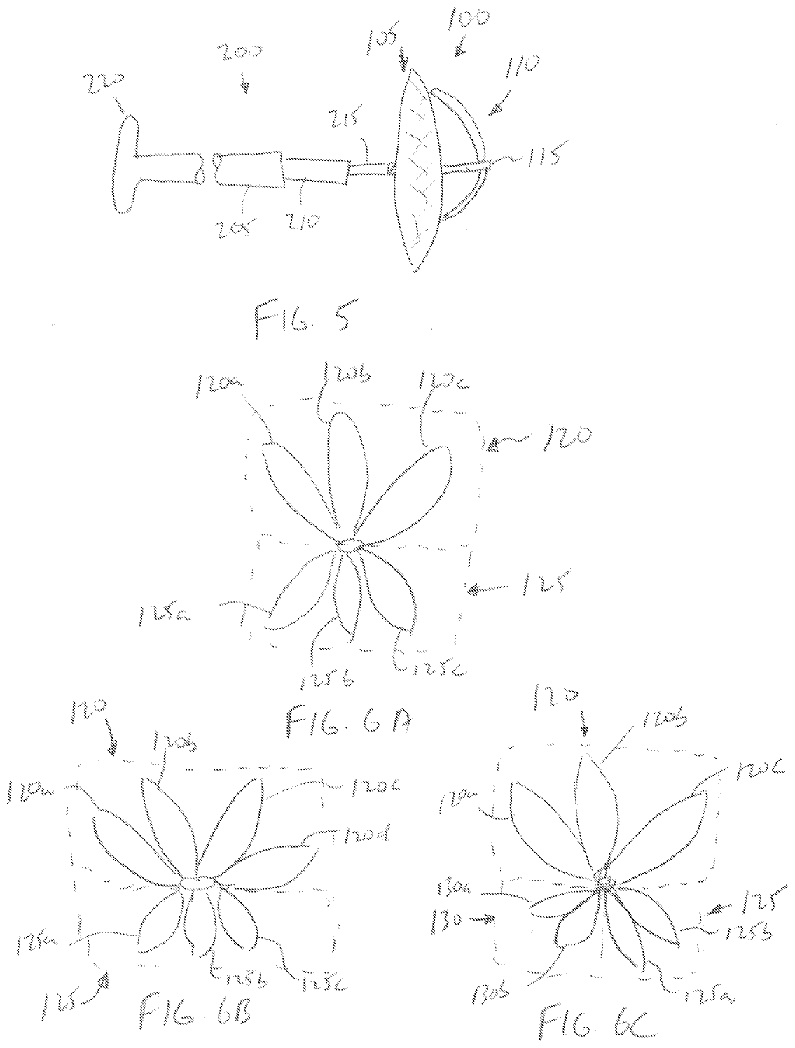

FIGS. 4A and 4B show the closure implant 100 coupled to the distal end of a delivery system 200. The closure implant 100 can be delivered to the hole in the septum, such as an atrial septal defect 70 (FIG. 4A) or a patent foramen ovale 60 (FIG. 4B), to prevent blood from flowing from the right atrium 15 to the left atrium 25.

The asymmetrical petal anchor 110 is positioned in the left atrium 25 side of the patent foramen ovale 60 or atrial septal defect 70 and the braided disk 105 is positioned on the right atrium 15 side. Once in place, the braided disk 105 and the petal anchor 110 compress against each side of the septum 35 to close the patent foramen ovale 60 or atrial septal defect 70 and fluidly seal the right atrium 15 from the left atrium 25 to reduce risk of clot formation and migration that might otherwise result.

FIG. 5 shows the closure implant 100 coupled to the distal end of a delivery system 200. The closure implant 100 can be delivered to the patent foramen ovale 60 or atrial septal defect 70 in a surgical procedure or via the catheter-based delivery system 200 in an interventional procedure. The delivery system 200 may include:

Steerable Introducer Sheath with Dilator 205
Short Steering section for up to 30-degree curve
PTFE Lined
Polyethylene dilator

Inner Steerable Sheath 210
180-degree steering section
PTFE Lined

Therapy “depth” Catheter 215
Braided, multi-durometer, no ptfe shaft
Connect/disconnect feature

Handle 220

The steerable introducer sheath with dilator includes an internal lumen that is configured and dimensioned to slidably receive the inner steerable sheath. The inner steerable sheath includes an internal lumen that is configured and dimensioned to slidably receive the catheter. The catheter is releasably coupled to the implant at the connect/disconnect feature to deliver the closure implant 100 to the patent foramen ovale 60 or atrial septal defect 70. Once the closure implant 100 deployed, the connect/disconnect feature is disconnected and withdrawn.

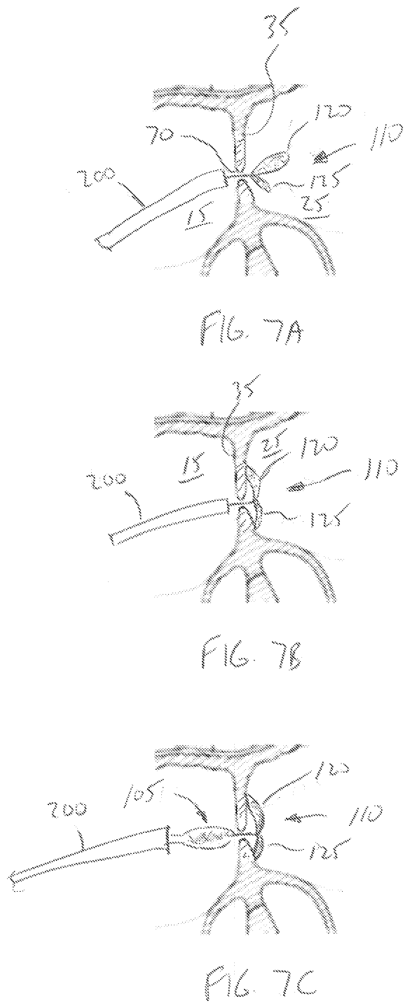

FIG. 6A-6C shows various embodiments of a pedal anchor 110 having petal groups with asymmetrical petals. For example, FIG. 6A shows one embodiment of a petal anchor 115 having two asymmetrical petal groups 120, 125 with the same number of petals: a first petal group 120 with three petals 120a, 120b, 120c and a second petal group 125 with three petals 125a, 125b, 125c. The petals of the first petal group 120 being larger than the petals of the second petal group 125. FIG. 6B shows another embodiment of a petal anchor 115 having two asymmetrical petal groups 120, 125 with different number of petals: a first petal group 120 with four petals 120a, 120b, 120c, 120d and a second petal group 125 with three petals 125a, 125b, 125c. The petals of the first petal group 120 being larger than the petals of the second petal group 125. FIG. 6C shows another embodiment of a petal anchor 115 having three asymmetrical petal groups 120, 125, 130 with different number of petals: a first petal group 120 with three petals 120a, 120b, 120c; a second petal group 125 with two petals 125a, 125b, and a third petal group 130 with two petals 130a, 130. The petals of the first petal group 120 being larger than the petals of the second petal group 125, and the petals of the second petal group 125 being larger than the petals of the third petal group 130.

The petals are made from a shape-memory wire, such as nitinol wire, with a cover material that is shaped like a flower petal. The petals may be pre-shaped in an expanded configuration to form the petal groups of the petal anchor. In the embodiments shown in the figures, the petals in the expanded configuration have a rearward curvature that is designed to engage or contact the wall of the septum around the hole to resist pull-out. The rearward curvature may look hook-shaped. The shape-memory material allows the petals to be delivered in a collapsed or compressed configuration within a delivery system or sheath. Then once delivered and released through the distal end of the delivery system or sheath, the shape-memory wire self-expands the petals to their expanded configurations into the expanded configuration and curve back proximally to engage the septum around the hole.

Prior to engagement with the septum, the petals may be torqued or rotated to a desired position. The petals may also be manipulated to change the size or shape by manipulating the wire. The petal wires in a petal group may be linked so that all the petals change at the same time, or each petal may have a separate wire to be done individually. Having adjustable petal wires for each petal segment allows individual adjustability of the petal groups to accommodate the anatomy or placement requirements of the implant. For example, the implant may be torqued or rotated so the petals of the petal groups lie relatively flat around patent foramen ovale 60 or atrial septal defect 70 and avoid defects or structures around the patent foramen ovale 60 or atrial septal defect 70.

In some embodiments that petals include petal wires that extend proximally and be manipulated to change the size and/or shape of the petal. Each petal wire may be manipulated separately to change size and/or shape, or the petals in each petal group may be linked to manipulate all the petals in the petal group at the same time. The adjustable petal wires for each petal allows adjustability of the petals to accommodate the anatomy or placement requirements of the implant. For example, the size of the petal may be changed to engage or avoid heart structures or defects in the septum wall.

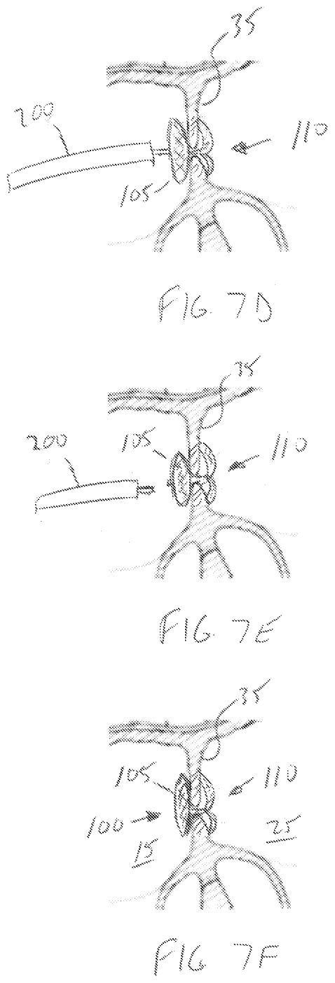

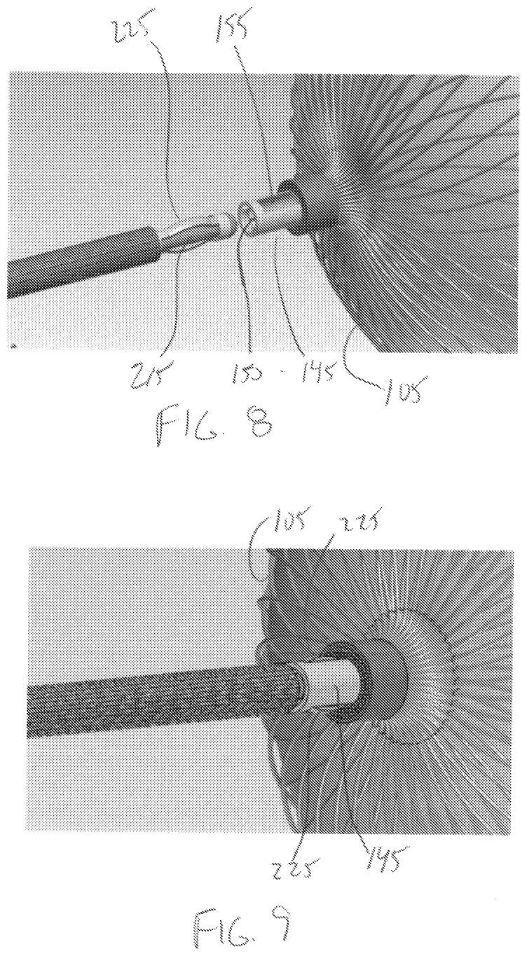

FIGS. 7A-7F show one embodiment of a closure implant 100 that is configured to close an opening 70 in the septum 35. The closure implant 100 is designed to be delivered within a lumen of a delivery system 200 in a compressed or delivery configuration. When the closure implant 100 is loaded into the lumen of the delivery system 200, the braided disk 105 and petal groups 120, 125 are compressed from their naturally expanded configuration to a compressed delivery configuration. The closure implant 100 is designed to be delivered from a distal end 115a of the delivery system 200.

FIG. 7A shows the distal end of the delivery system 200 positioned proximate the hole 70. The first petal group 120 and the second petal group 125 of the petal anchor 110 are delivered distally from the distal end, through the hole 70 and into the left atrium 25. Once released from the delivery system 200, the first petal group 120 and second petal group 125 begin to self-expand.

If the petal groups 120, 125 have a problem going through the hole 70, or they expand too quickly before they can go through the hole 70, it may be desirable to advance the distal end of the delivery system 200 through the hole 70 into the left atrium 25 and deliver the petal anchor 110 directly to the distal side of the septum 35.

FIG. 7B shows the first petal group 120 and the second petal group 125 after self-expanding to their naturally expanded configuration, curving proximally and contacting the wall of the septum 35.

FIG. 7C shows the delivery system 200 being retracted proximally from the hole 70 to advance the braided disk 105 from the distal end in the right atrium 15. Once released from the delivery system 200, braided disk 105 begins to self-expand and positioned on the proximal side of the septum 35, opposite the petal anchor 110.

FIG. 7D shows the braided disk 105 after it has expanded from the compressed delivery configuration to the naturally expanded configuration, which should be larger than the hole 70. When in position, the braided disk 105 is on the proximal side covering the hole 70 and the petal anchor 110 is on the distal side 70. If needed, the petal wires can be pulled proximally to compress the braided disk 105 against the hole 79 and fluidly seal the hole 70 to prevent blood flow from the right atrium 15 to the left atrium 25.

FIG. 7E shows the closure implant 100 in place and the delivery system 200 being disconnected.

FIG. 7F shows the closure implant 100 in place fluidly sealing the hole 70 between the right atrium 15 and left atrium 25.

FIG. 8 is a perspective view showing the components of the connect/disconnect feature that are configured to couple the shaft 215 of a delivery system 200 with the shaft 120 of the closure implant 100. The coupling of the shafts 120, 215 allow the delivery system 200 to torque the closure implant 100 and have zero release force for the closure implant 100 when the components are uncoupled. This provides the ability to torque the asymmetrical petals of the closure implant 100 for optimum seal and optimum durability.

The proximal end of the closure implant 100 includes a coupler 145 having a central opening 150 and slots 155. The distal end of the shaft 215 includes one or more engagement arms 225 having springlike properties that allow them to deflect and spring back to their original position.

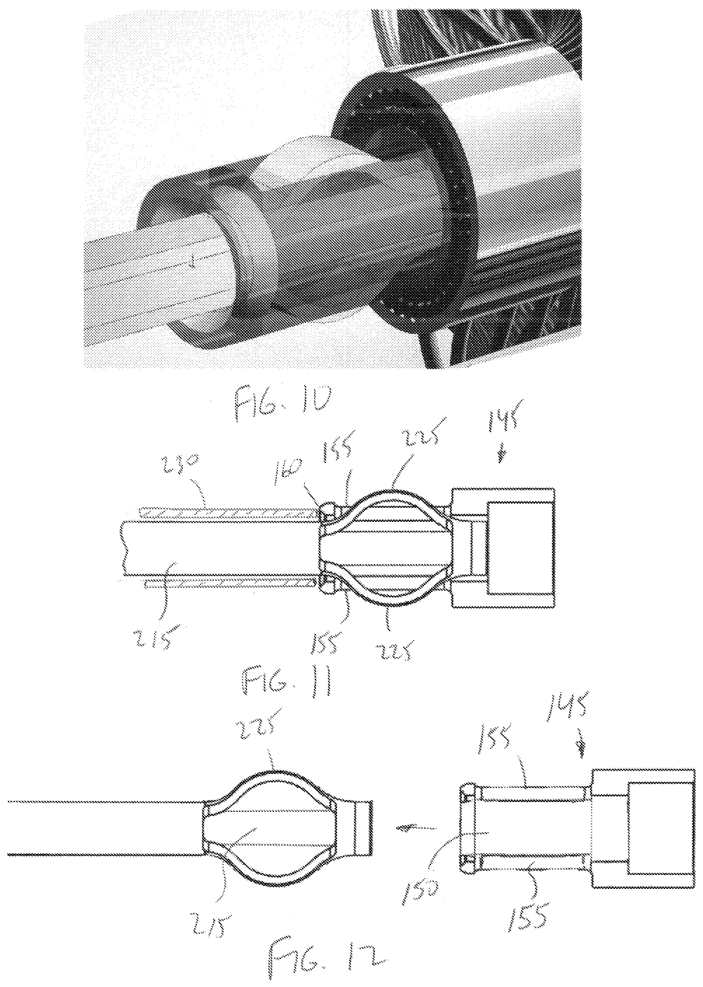

FIG. 9 is perspective view showing the closure implant 100 coupled to the delivery system 200. The distal end of the shaft 215 is sized for insertion into the central opening 150. During insertion, a curved distal portion of the engagement arms/springs 225 contacts the coupler 145 and deflects inwardly into the central opening 150 until the engagement arms/springs 225 line up with the slots 155. Then the spring arms 225 return to their original shape and engage the slots 155. When the engagement arms/springs 225 are coupled with the slots 155, the delivery system 200 may rotate or torque the closure implant 100.

FIG. 10 is perspective view of the connect/disconnect feature of the invention showing the delivery system 200 coupled to the closure implant 100. The connect/disconnect feature shows the distal end of the Inner Steerable Sheath 210 and engagement arms 225 within the central opening 150 of the coupler 145 with the engagement arms 225 positioned within the slots 155. The coupler 145 is semi-transparent to show more details of the connection.

FIG. 11 is a sectional view showing the engagement of the distal end of the shaft 215 and engagement arms/springs 225 coupled to the coupler 145 and slots 155. To disconnect the delivery system 200 from the closure implant 100, a removal tube 230 is distally slid over the shaft 215 until it slides over 155 and compressed the engagement arms/springs 225 and stops the proximal end of the coupler 145. Once the engagement arms/springs 225 are “sheathed”, the shaft 215 is advanced distally, to aid in straightening the engagement arms/spring. Both of these actions cause the engagement arms/springs 225 to deflect inward, allowing removal of the outer shaft from the central opening of the coupler 145.

FIG. 12 is a sectional view showing the delivery system 200 disengaged from the closure implant 100.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. An implant for sealing a patent foramen ovale, comprising:
a body having a proximal end and a distal end;
a disk coupled near the proximal end of the body, the disk configured to seal the patent foramen ovale in the right atrium; and
an anchor coupled near the proximal end of the body, the anchor comprising asymmetrical pedals of two or more sizes having a rearward curvature configured to engage the septum proximate patent foramen ovale in the left atrium.

2. The implant of claim 1, wherein the asymmetrical petals are configured to accommodate the anatomy of the septum wall.

3. The implant of claim 1, wherein the asymmetrical pedals are self-expanding.

4. The implant of claim 3, wherein the asymmetrical petals are made from a shape memory material that is pre-shaped in a desired rearward curvature shape.

5. The implant of claim 3, wherein the asymmetrical petals are configured to be collapsed or compressed to a delivery shape for delivery and self-expand to the desired curvature shape at the delivery site.

6. The implant of claim 1, wherein the asymmetrical pedals include multiple groups of pedals on opposite sides of the body.

7. The implant of claim 6, wherein the groups of asymmetrical pedals include two or more petals per group.

8. The implant of claim 1, wherein the asymmetrical pedals are adjustable in size.

9. The implant of claim 8, wherein the asymmetrical pedals are adjustable either individually or in groups.

10. The implant of claim 1, wherein the disk is self-expanding. The implant of claim 10, wherein the expandable disk is a mesh structure made from a shape memory material that is pre-shaped in a desired disk shape.

12. The implant of claim 1, wherein the disk is covered with a covering material.

13. The implant of claim 10, wherein the expandable disk is configured to be collapsed or compressed to a delivery shape for delivery and self-expand to the desired disk shape at the delivery site.

14. The implant of claim 1, wherein the proximal end of the body includes a coupler configured to releasably couple with a distal shaft of a delivery catheter.

15. The implant of claim 14, wherein the coupler includes one or more slots and the distal shaft end of the shaft include or more engagement arms, the one or more engagement arms being configured to couple with one or more slots to allow the distal shaft of the delivery system to rotate or torque the implant during delivery.

16. An implant for sealing a patent foramen ovale, comprising:
a body having a proximal end and a distal end;
a self-expanding disk with a covering material coupled to the body near the proximal end;
a self-expanding anchor coupled near the proximal end of the body, the anchor comprising asymmetrical pedals of two or more sizes having a rearward curvature configured to engage the septum proximate patent foramen ovale in the left atrium with enough force to resist pull-out.

17. The implant of claim 16, wherein the petals include asymmetrical proximal petals of two different size pedals coupled to the body on opposing sides near the proximal end.

18. The implant of claim 16, wherein the asymmetrical petals are configured to accommodate the anatomy of the septum wall.

19. The implant of claim 16, wherein the anchor is configured to be collapsed or compressed in a delivery configuration for delivery of the implant to the septum wall, and then self-expand after delivery.

20. An implant for sealing a patent foramen ovale, comprising:
a body having a proximal end and a distal end;
a self-expanding disk with a covering material coupled to the body near the proximal end;
an expandable proximal anchor having proximal asymmetrical proximal petals of two different size pedals coupled to the body on opposing sides near the proximal end, the proximal asymmetrical petals having a rearward curvature configured to engage the septum proximate patent foramen ovale in the left atrium with enough force to resist pull-out; and

21. The implant of claim 20, wherein the asymmetrical proximal and the asymmetrical distal petals are configured to accommodate the anatomy of the septum proximate patent foramen ovale in the left atrium with enough force to resist pull-out.

22. The implant of claim 20, wherein the disk and anchor are configured to be collapsed or compressed in a delivery configuration for delivery of the implant to the patent foramen ovale and then self-expand after delivery.
